SEQUENCE LISTING (7 sequences)

SEQ ID NO: 1 - protein, 9 aa, Artificial
  VHH CDRH1
GHFSNLAVN

SEQ ID NO: 2 - protein, 17 aa, Artificial
  VHH CDRH2
GILWSGGSTFYADSVKG

SEQ ID NO: 3 - protein, 3 aa, Artificial
  VHH CDRH3
GTN

SEQ ID NO: 4 - protein, 111 aa, Artificial
  VHH domain
EVQLVESGGGLVQPGGSLRLSCAASGHFSNLAVNWFRQAPGKERELVAGILWSGGSTFYADSVKGRFTISRGNAENMLYLQMNSLRAEDTAVYYCNTGTNWGQGTLVTVSS

SEQ ID NO: 5 - protein, 21 aa, Artificial
  Linker
GGGGGSGGGGSGGGGSGGGGS

SEQ ID NO: 6 - protein, 136 aa, Artificial
  TGF beta RII
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 7 - protein, 498 aa, Artificial sequence
  Synthetic
EVQLVESGGGLVQPGGSLRLSCAASGHFSNLAVNWFRQAPGKERELVAGILWSGGSTFYADSVKGRFTISRGNAENMLYLQMNSLRAEDTAVYYCNTGTNWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

(19) United States
(12) Patent Application Publication
(10) Pub. No.: US 2023/0035169 A1
(43) Pub. Date: Feb. 2, 2023

(54) BI-FUNCTIONAL ANTIBODY AGAINST PD-L1 AND TGF-Beta

(71) Applicant: WUXI BIOLOGICS IRELAND LIMITED, Dublin (IE)

(72) Inventors: Yunying CHEN, Shanghai (CN); Jing LI, Shanghai (CN)

(73) Assignee: WUXI BIOLOGICS IRELAND LIMITED, Dublin (IE)

(21) Appl. No.: 17/784,617

(22) PCT Filed: Dec. 11, 2020
(86) PCT No.: PCT/CN2020/135928

§ 371 Date: Jun. 10, 2022

(30) Foreign Application Priority Data
Dec. 11, 2019 (CN) PCT/CN2019/124535

(51) Int. Cl. C07K 16/28; C12N 15/63; A61P 35/00

(52) U.S. Cl. CPC C07K 16/2827; C12N 15/63; A61P 35/00; C07K 2317/565; C07K 2317/52

(57) ABSTRACT

Provided is a polypeptide, comprising, from N-terminus to C-terminus, a) at least a variable region of a heavy chain of a heavy-chain antibody (VHH domain) that binds protein Programmed Death Ligand 1 (PD-L1), operably linked to IgG Fc binding domain; and b) human TGFβRII, or a fragment thereof capable of binding TGFβ. An antibody comprises the said polypeptide, the amino acid sequences of the said antibody, cloning or expression vectors, cells and methods for expressing or isolating the antibodies are further provided. Therapeutic compositions comprising the said antibodies, and the methods for treating cancers and other diseases with the bispecific antibodies are also provided.

### TECHNICAL FIELD

The present invention relates to bi-functional antibodies. Moreover, the invention provides a polynucleotide encoding the antibodies, a vector comprising said polynucleotide, a host cell, a process for the production of the antibodies and immunotherapy in the treatment of cancer, infections or other human diseases using the bi-functional antibodies.

### BACKGROUND OF THE INVENTION

Bi-functional antibodies can bind to two different targets or two different epitopes on a target, creating additive or synergistic effects superior to the effects of individual monospecific antibodies or target binding modules. VHH, the Variable domain of Heavy chain only antibodies discovered in Camelidae, is considered the smallest naturally derived antigen-binding fragments (12-15 kDa) that can be isolated from a full-sized immunoglobulin. Due to its small size and high antigen-binding affinity, good tumor penetration of VHH is expected. In addition, VHH has high stability and simplicity in recombinant expression. Therefore, VHH exhibits profound advantage over conventional antibody for the application of bi-functional antibody.

Programmed cell death ligand 1 (PD-L1, B7-H1, CD274) is a member of the immunoglobulin superfamily induced on a variety of cell types in lymphoid and peripheral tissues. Programmed cell death-1 (PD-1, CD279) is a member of CD28 family expressed on activated T cells and other immune cells. The major role of PD-1 pathway is to tune down immune response in tissues and organs. It is found that cancer cells are capable of evading immune destruction by upregulating PD-1/PD-L1 pathway in the tumor microenvironment [Boussiotis 2016 N Engl J Med].

TGF-β can promote tumor progression and facilitate tumor immune evasion through its effects on the innate and adaptive immune systems. The three TGF-β isoforms, TGF-β1, TGF-β2, and TGF-β3, are highly expressed in many tumor types, and their serum concentrations correlate with poor clinical outcome. TGF-β functions as an autocrine or paracrine signal within the tumor microenvironment, where it promotes tumor progression via stromal modification, angiogenesis, and induction of epithelial-mesenchymal transition (EMT). TGF-β signaling in myeloid cells is also critical in driving metastasis. In addition, TGF-β1 can directly inhibit T cell division and acquisition of effector function and natural killer (NK) cell function.

Antibodies targeting immune checkpoints are emerging as potent and viable cancer therapies, but not all patients respond to these as single agents. Concurrently targeting additional immunosuppressive pathways is a promising approach to enhance immune checkpoint blockade, and bifunctional molecules designed to target two pathways simultaneously may provide a strategic advantage over the combination of two single agents.

This invention aims to develop a novel and potent antitumor therapeutics, which is comprised of VHH against PD-L1, devoid of light chain and fused with TGFβ Receptor II ECD (extracellular domain).

### SUMMARY OF THE INVENTION

The present invention is based on the discovery that a bifunctional polypeptide containing at least portion of TGFβ Receptor II (TGFβRII) that is capable of binding TGFβ and VHH domain that binds to an immune checkpoint protein such as human, monkey, or mouse protein Programmed Death Ligand 1 (PD-L1) can be an effective anti-tumor and anti-cancer therapeutic. The protein can exhibit a synergistic effect in cancer treatment, as compared to the effect of administering the two agents separately.

Accordingly, in a first aspect, the present invention provides a polypeptide, comprising, from N-terminus to C-terminus, at least a variable region of a heavy chain of a heavy-chain antibody (VHH domain) that binds protein Programmed Death Ligand 1 (PD-L1), operably linked to IgG Fc binding domain; and human TGFβRII (e.g., a human TGFβRII extra-cellular domain (ECD), SEQ ID No: 6), or a fragment thereof capable of binding TGFβ.

In one embodiment, TGFβ is selected from the group consisting of TGFβ1, TGFβ2, and TGFβ3.

In one embodiment, the V-H11 domain comprises one or more heavy chain CDRs (CDRHs) selected from at least one of the group consisting of:

(a) a CDRH1 with at least 70%, sequence identity to a CDRH1 as depicted in SEQ ID NO: 1;

(b) a CDRH2 with at least 70%, sequence identity to a CDRH2 as depicted in SEQ ID NO: 2; and

(c) a CDRH3 with at least 70%, sequence identity to a CDRH3 as depicted in SEQ ID NO: 3.

In a specific embodiment, VHH domain comprises one or more heavy chain CDRs (CDRHs) selected from at least one of the group consisting of:

(a) a CDRH1 as depicted in SEQ ID NO: 1 or a CDRH1 that differs in amino acid sequence from the CDRH1 by an amino acid addition, deletion or substitution of not more than 2 amino acids;

(b) a CDRH2 as depicted in SEQ ID NO: 2 or a CDRH2 that differs in amino acid sequence from the CDRH2 by an amino acid addition, deletion or substitution of not more than 2 amino acids; and

(c) a CDRH13 as depicted in SEQ ID NO: 3 or a CDRH3 that differs in amino acid sequence from the CDRH3 by an amino acid addition, deletion or substitution of not more than 2 amino acids.

In a specific embodiment, the VHH domain comprises one or more heavy chain CDRs (CDRHs) selected from at least one of the group consisting of:

(a) a CDRH1 comprising or consisting of SEQ ID NO: 1;

(b) a CDRH2 comprising or consisting of SEQ ID NO: 2; and

(c) a CDRH3 comprising or consisting of SEQ ID NO: 3.

In a specific embodiment, the VHH domain comprises:

(a) the amino acid sequence of SEQ ID NO: 4;

(b) an amino acid sequence at least 85%, 90%, 95%, or 99% identical to SEQ ID NO: 4; or

(c) an amino acid sequence with addition, deletion and/or substitution of one or more amino acids compared with SEQ ID NO: 4.

The polypeptide may further include an amino acid linker connecting the C-terminus of the VHH domain or IgG Fc binding domain to the N-terminus of the human TGFβRII or fragment thereof; the linker comprises:

(a) the amino acid sequence of SEQ ID NO: 5;

(b) an amino acid sequence at least 85%, 90%, 95%, or 99% identical to SEQ ID NO: 5; or

(c) an amino acid sequence with addition, deletion and/or substitution of one or more amino acids compared with SEQ ID NO: 5.

In one embodiment, the human TGFβRII, or a fragment thereof comprises:

(a) the amino acid sequence of SEQ ID NO: 6;

(b) an amino acid sequence at least 85%, 90%, 95%, or 99% identical to SEQ ID NO: 6; or

(c) an amino acid sequence with addition, deletion and/or substitution of one or more amino acids compared with SEQ ID NO: 6.

The TGFβRII, or a fragment thereof may retain at least 0.1%, 0.5%, 1%, 5% 10%, 25%35%, 50%, 75%, 90%, 95%, or 99% of the TGFβ-binding activity of the wild-type sequence.

In one embodiment, the polypeptide may include

(a) the amino acid sequence of SEQ ID NO: 7;

(b) an amino acid sequence at least 85%, 90%, 95%, or 99% identical to SEQ ID NO: 7; or

(c) an amino acid sequence with addition, deletion and/or substitution of one or more amino acids compared with SEQ ID NO: 7.

The invention also provides an antibody or antigen binding fragment thereof, wherein the antibody or antigen binding fragment thereof comprises two of foresaid polypeptide.

The sequences of the polypeptide or antibodies are shown is Table 1:

TABLE 1
The sequences of the antibodies.

| WT1126-U15T1.G1.uIgG1 | | SEQ ID NO | Amino acid sequence |
|---|---|---|---|
| VHH | CDRH1 | 1 | GHFSNLAVN |
| | CDRH2 | 2 | GILWSGGSTFYADSVKG |
| | CDRH3 | 3 | GTN |
| | VHH | 4 | EVQLVESGGGLVQPGGSLRLSCAASGHFSNLAVNWFRQ |
| | | | APGKERELVAGILWSGGSTFYADSYKGRFTISRGNAENM |
| | | | LVLQMNSLRAEDTAVYYCNTGTNWGQGTLVTVSS |
| Linker | | 5 | GGGGGSGGGGSGGGGSGGGGS |
| TGFβRII extra-cellular | | 6 | IPPHVQKSVNNDMIVTDNNGAVKFPQLCKFCDVRFSTC |
| domain | | | DNQKSCMSNCSITSICEKPQEVCVAVWRKNDENITLETV |
| | | | CHDPKLPYHDFILEDAASPKCIMKEKKKPGETFFMCSCS |
| | | | SDECNDNIIFSEEYNTSNPD |
| Full length | | 7 | EVQLVESGGGLVQPGGSLRLSCAASGHFSNLAVNWFRQ |
| | | | APGKERELVAGILWSGGSTFYADSVKGRFTISRGNAENM |
| | | | LYLQMNSLRAEDTAVYYCNTGTNWGQGTLVTVSSEPKS |
| | | | CDKTHTCPPCPAPELLGGPSVFLFPPKPKDTLMISRTPEV |
| | | | TCVVVDVSHEDPEVKFNWYVDGVEVHNAKTKPREEQ |
| | | | YNSTYRVVSVLTVLHQDWLNGKEYKCKVSNKALPAPIE |
| | | | KTISKAKGQPREPQVYTLPPSREEMTKNQVSLTCLVKGF |
| | | | YPSDIAVEWESNGQPENNYKTTPPVLDSDGSFFLYSKLT |
| | | | VDKSRWQQGNWSCSVMHEALHNHYTQKSLSLSPGGG |
| | | | GGSGGGGSGGGGSGGGGSIPPHVQKSVNNDMIVTDNN |
| | | | GAVKFPQLCKFCDVRFSTCDNQKSCMSNCSITSICEKPQ |
| | | | EVCVAVWRKNDENITLETVCHDPKLPYHDFILEDAASPK |
| | | | CIMKEKKKPGETFFMCSCSSDECNDNIIFSEEYNTSNPD |

In a further aspect, the invention provides a nucleic acid molecule comprising a nucleotide sequence encoding the polypeptide described above or antibody or antigen binding fragment thereof described above.

The invention provides a cloning or expression vector comprising the nucleic acid molecule encoding the polypeptide described above or antibody or antigen binding fragment thereof described above.

The invention also provides a cell comprising the nucleic acid described above or one or more cloning or expression vectors described above.

In yet another aspect, the invention provides a process, comprising culturing the cell of the invention and isolating the polypeptide or antibody.

In a further aspect, the invention provides pharmaceutical composition comprising the polypeptide, or antibody, or antigen binding fragment of said antibody in the invention, and one or more of a pharmaceutically acceptable excipient, a diluent or a carrier.

The invention also provides the polypeptide described above or the antibody described above, for use in treating tumor or growth of tumor cells in a subject.

The invention also provides a method of treating tumor or inhibiting growth of tumor cells in a subject, comprising administering to the subject a therapeutically effective amount of the polypeptide or the antibody described above.

The invention also provides use of the polypeptide or the antibody described above in the manufacture of a medicament for treating tumor or inhibiting growth of tumor cells in a subject.

In the present invention, the tumor is selected from the group consisting of colorectal, breast, ovarian, pancreatic, gastric, prostate, renal, cervical, myeloma, lymphoma, leukemia, thyroid, endometrial, uterine, bladder, neuroendocrine, head and neck, liver, nasopharyngeal, testicular, small cell lung cancer, non-small cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodysplastic syndromes.

The features and advantages of this invention

A bi-functional molecular against both PD-L1 and TGFβ pathways may provide several benefits in cancer therapy. Compared with anti-PD-L1 therapy, the bi-functional antibody may increase the response rate.

### BRIEF DESCRIPTION OF DRAWINGS

FIG. 1 shows the schematic diagram of WT1126-U15T1. G1-1. uIgG1.

FIG. 2 shows the binding to immobilized human TGF-β1, TGFβ2 and TGF-β3 by ELISA.

FIG. 3 shows the binding to soluble TGF-β2 by ELISA.

FIG. 4 shows the binding to cell surface human PD-L1 by FACS.

FIG. 5 shows the binding to cell surface cyno PD-L1 by FACS.

FIG. 6 shows the binding to cell surface mouse PD-L1 by FACS.

FIG. 7 shows the dual target binding ELISA immobilized with TGF-β1.

FIG. 8 shows the dual target binding ELISA immobilized with human PD-L1.

FIG. 9 shows the PD-1/PD-L1 blockade by competition FACS.

FIG. 10 shows the TGF-β1 signaling blockade in a RGA assay.

FIG. 11 shows the PD-1/PD-L1 signaling blockade in a RGA assay.

FIG. 12A shows IL-2 secretion in Human CD4+ T cell allo-MRL assay. FIG. 12B shows IFN-γ secretion in Human CD4+ T cell allo-MRL assay.

FIG. 13 shows serum stability test.

FIG. 14A shows body weight growth curve of CT26 model; FIG. 14B shows tumor growth curve of CT26 model.

### DETAILED DESCRIPTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

The articles “a”, “an”, and “the” are used herein to refer to one or to more than one (i.e., to at least one) of the grammatical object of the article. By way of example, “a polypeptide complex” means one polypeptide complex or more than one polypeptide complex.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

Throughout this disclosure, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The terms “polypeptide”, “peptide”, and “protein” are used interchangeably herein to refer to a polymer of amino acid residues, or an assembly of multiple polymers of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds that have the same basic chemical structure as a naturally occurring amino acid, i.e., an alpha-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. An alpha-carbon refers to the first carbon atom that attaches to a functional group, such as a carbonyl. A beta-carbon refers to the second carbon atom linked to the alpha-carbon, and the system continues naming the carbons in alphabetical order with Greek letters. Amino acid mimetics refers to chemical compounds that have a structure that is different from the general chemical structure of an amino acid, but that functions in a manner similar to a naturally occurring amino acid. The term “protein” typically refers to large polypeptides. The term “peptide” typically refers to short polypeptides. Polypeptide sequences are usually described as the left-hand end of a polypeptide sequence is the amino-terminus (N-terminus); the right-hand end of a polypeptide sequence is the carboxyl-terminus (C-terminus). “Polypeptide complex” as used herein refers to a complex comprising one or more polypeptides that are associated to perform certain functions. In certain embodiments, the polypeptides are immune-related.

The term “fragment of TGFβRII” refer to any portion of a sequence substantially identical to SEQ ID NO: 6 that is at least 20 (e.g., at least 30, 40, 50, 60, 70, 80, 90, 100, 110, 120, 130, 140, 150, 160, 175, or 200) amino acids in length that retains at least some of the TGFβ-binding activity (e.g., at least 0.1%, 0.5%, 1%, 5%, 10%, 25%, 35%, 50%, 75%, 90%, 95%, or 99%) of the wild-type receptor or of the corresponding wild-type fragment. Typically such fragment is a soluble fragment. An exemplary such fragment is a TGFβRII extra-cellular domain having the sequence of SEQ ID NO: 6.

The term “substantially identical” refers to a polypeptide exhibiting at least 50%, desirably 60%, 70%, 75%, or 80%, more desirably 85%, 90%, or 95%, and most desirably 99% amino acid sequence identity to a reference amino acid sequence. The length of comparison sequences will generally be at least 10 amino acids, desirably at least 15 contiguous amino acids, more desirably at least 20, 25, 50, 75, 90, 100, 150, 200, 250, 300, or 350 contiguous amino acids, and most desirably the full-length amino acid sequence.

The term “antibody” as used in this disclosure, refers to an immunoglobulin or a fragment or a derivative thereof, and encompasses any polypeptide comprising an antigen-binding site, regardless whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. The term “antibody” also includes antibody fragments such as scFv, dAb, bispecific antibodies comprises a first VHH domain and TGFβRII or fragment thereof fusion, and other antibody fragments that retain antigen-binding function, i.e., the ability to bind PD-L1.

An antigen-binding fragment typically comprises an antibody light chain variable region (VL) and an antibody heavy chain variable region (VH), however, it does not necessarily have to comprise both. The VH and VL regions may be further subdivided into highly variable regions known as complementary decision regions (CDRs), interspersed with more conservative regions known as framing regions (FRs). The numbering scheme for all CDR definitions in the present invention is determined by the Kabat numbering system. A so-called Nano antibody fragment consists only of a VHH domain, CH2 and CH3 domain, but still retains some antigen-binding function of the intact antibody.

“Fc” with regard to an antibody refers to that portion of the antibody consisting of the second (CH2) and third (CH3) constant regions of a first heavy chain bound to the second and third constant regions of a second heavy chain via disulphide bonding. The Fc portion of the antibody is responsible for various effector functions such as ADCC, and CDC, but does not function in antigen binding.

“CH2 domain” as used herein refers to includes the portion of a heavy chain molecule that extends, e.g., from about amino acid 244 to amino acid 360 of an IgG antibody using conventional numbering schemes (amino acids 244 to 360, Kabat numbering system; and amino acids 231-340, EU numbering system; see Kabat, E., et al., U.S. Department of Health and Human Services, (1983)).

The “CH3 domain” extends from the CH2 domain to the C-terminus of the IgG molecule and comprises approximately 108 amino acids. Certain immunoglobulin classes, e.g., IgM, further include a CH4 region.

The term “antigen-binding moiety” as used herein refers to an antibody fragment formed from a portion of an antibody comprising one or more CDRs, or any other antibody fragment that binds to an antigen but does not comprise an intact native antibody structure.

The terms “Programmed Death ligand 1”, “PD ligand 1”, “PD-L1”, “PD L1”, “B7 homolog i”, “B7-H1”, “B7 H1”, “CD274” are used interchangeably, and include variants, isoforms, species homologs of human PD-L1, and analogs having at least one common epitope with PD-L1.

The term “homolog” and “homologous” as used herein are interchangeable and refer to nucleic acid sequences (or its complementary strand) or amino acid sequences that have sequence identity of at least 70% (e.g., at least 70%, 75%, 80%, 85%, 88%, 90%, 91%, 92%, 93%, 94%, 95%, 96%, 97%, 98%, 99%) to another sequences when optimally aligned.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215: 403-410 (1990); Stephen F. et al., Nucleic Acids Res., 25: 3389-3402 (1997)), ClustalW2 (available on the website of European Bioinformatics Institute, see also, Higgins D. G et al., Methods in Enzymology, 266: 383-402 (1996); Larkin M. A. et al., Bioinformatics (Oxford, England), 23 (21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm.

The term “binding” or “binds” as used herein refers to a non-random binding reaction between two molecules, such as for example between an antibody and an antigen. In certain embodiments, the polypeptide complex and the bispecific polypeptide complex provided herein specifically bind an antigen with a binding affinity (K D) of ≤10⁻⁶ M (e.g., ≤5×10⁻⁷ M, ≤2×10⁻⁷ M, ≤10⁻⁷M, ≤5×10⁻⁸ M, ≤2×10⁻⁸M, ≤10⁻⁸M, ≤5×10⁻⁹ M, ≤2×10⁻⁹ M, ≤10⁻⁹ M, or ≤10⁻¹⁰ M). K D as used herein refers to the ratio of the dissociation rate to the association rate (k off/k on), may be determined using surface plasmon resonance methods for example using instrument such as Biacore.

The term “operably link” or “operably linked” refers to a juxtaposition, with or without a spacer or linker, of two or more biological sequences of interest in such a way that they are in a relationship permitting them to function in an intended manner. When used with respect to polypeptides, it is intended to mean that the polypeptide sequences are linked in such a way that permits the linked product to have the intended biological function. For example, an antibody variable region may be operably linked to a constant region so as to provide for a stable product with antigen-binding activity. The term may also be used with respect to polynucleotides. For one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide.

The term “mutation” or “mutated” with regard to amino acid residue as used herein refers to substitution, insertion, or addition of an amino acid residue.

Format

The present invention provides a polypeptide, comprising, from N-terminus to C-terminus, at least a variable region of a heavy chain of a heavy-chain antibody (VHH domain) that binds protein Programmed Death Ligand 1 (PD-L1), operably linked to IgG Fc binding domain; and human TGFβRII (e.g., a human TGFβRII extra-cellular domain (ECD)), or a fragment thereof capable of binding TGFβ. TGFβ is selected from the group consisting of TGFβ1, TGFβ2, and TGFβ3. The polypeptide may further include an amino acid linker connecting the C-terminus of the VHH domain or IgG Fc binding domain to the N-terminus of the human TGFβRII or fragment thereof. The invention also provides an antibody or antigen binding fragment thereof, wherein the antibody or antigen binding fragment thereof comprises two of foresaid polypeptide (FIG. 1).

In one embodiment, the VHH domain comprises one or more heavy chain CDRs (CDRHs) selected from at least one of the group consisting of:

(a) a CDRH1 with at least 70%, 80%, or 90%, sequence identity to a CDRH1 as depicted in SEQ ID NO: 1 (GHFSNLAVN);

(b) a CDRH2 with at least 70%, 80%, or 90%, sequence identity to a CDRH2 as depicted in SEQ ID NO: 2 (GILWSGGSTFYADSVKG); and

(c) a CDRH3 with at least 70%, 80%, or 90%, sequence identity to a CDRH3 as depicted in SEQ ID NO: 3 (GTN).

In a specific embodiment, VHH domain comprises one or more heavy chain CDRs (CDRHs) selected from at least one of the group consisting of:

(a) a CDRH1 as depicted in SEQ ID NO: 1 or a CDRH1 that differs in amino acid sequence from the CDRH1 by an amino acid addition, deletion or substitution of 1 or 2 amino acids;

(b) a CDRH2 as depicted in SEQ ID NO: 2 or a CDRH2 that differs in amino acid sequence from the CDRH2 by an amino acid addition, deletion or substitution of 1 or 2 amino acids; and

(c) a CDRH3 as depicted in SEQ ID NO: 3 or a CDRH3 that differs in amino acid sequence from the CDRH3 by an amino acid addition, deletion or substitution of 1 or 2 amino acids.

In a specific embodiment, the VHH domain comprises one or more heavy chain CDRs (CDRHs) selected from at least one of the group consisting of:

(a) a CDRH1 comprising or consisting of SEQ ID NO: 1;

(b) a CDRH2 comprising or consisting of SEQ ID NO: 2; and

(c) a CDRH3 comprising or consisting of SEQ ID NO: 3.

In a specific embodiment, the VHH domain comprises:

(a) the amino acid sequence of SEQ ID NO: 4

(EVQLVESGGGLVQPGGSLRLSCAASGHFSNLAVNWFRQAPGKERELVA
GILWSGGSTFYADSVKGRFTISRGNAENMLYLQMNSLRAEDTAVYYCNT
GTNWGQGTLVTVSS);

(b) an amino acid sequence at least 85%, 86%, 87%, 88%, 89%, 90%, 91%, 92%, 93%, 94%, 95%, 96%, 97%, 98%, or 99% identical to SEQ ID NO: 4; or

(c) an amino acid sequence with addition, deletion and/or substitution of one or more amino acids compared with SEQ ID NO: 4.

The polypeptide may further include an amino acid linker connecting the C-terminus of the VHH domain or IgG Fc binding domain to the N-terminus of the human TGFβRII or fragment thereof; the linker comprises:

(a) the amino acid sequence of SEQ ID NO: 5 (GGGGGSGGGGSGGGGSGGGGS);

(b) an amino acid sequence at least 85%, 86%, 87%, 88%, 89%, 90%, 91%, 92%, 93%, 94%, 95%, 96%, 97%, 98%, or 99% identical to SEQ ID NO: 5; or

(c) an amino acid sequence with addition, deletion and/or substitution of one or more amino acids compared with SEQ ID NO: 5.

In one embodiment, the human TGFβRII, or a fragment thereof comprises:

(a) the amino acid sequence of SEQ ID NO: 6

(IPPHVQKSVNNDMIVTDNNGAVKFPQLCKFCDVRFSTCDNQKSCMSNC
SITSICEKPQEVCVAVWRKNDENITLETVCHDPKLPYHDFILEDAASPK
CIMKEKKKPGETFFMCSCSSDECNDNIIFSEEYNTSNPD);

(b) an amino acid sequence at least 85%, 86%, 87%, 88%, 89%, 90, 91%, 92%, 93%, 94%, 95%, 96%, 97%, 98%, or 99% identical to SEQ ID NO: 6; or

(c) an amino acid sequence with addition, deletion and/or substitution of one or more amino acids compared with SEQ ID NO: 6.

The TGFβRII or fragment thereof may retain at least 0.1%, 0.5%, 1%, 5%, 10%, 25%, 35%, 50%, 75%, 90%, 95%, or 99% of the TGFβ-binding activity of the wild-type sequence.

In one embodiment, the polypeptide may include

(a) the amino acid sequence of SEQ ID NO: 7

(EVQLVESGGGLVQPGGSLRLSCAASGHFSNLAVNWFRQAPGKERELVA
GILWSGGSTFYADSVKGRFTISRGNAENMLYLQMNSLRAEDTAVYYCNT
GTNWGQGTLVTVSSEPKSCDKTHTCPPCPAPELLGGPSVFLFPPKPKDT
LMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
GGGGSGGGGSGGGGSGGGGSIPPHVQKSVNNDMIVTDNNGAVKFPQLCK
FCDVRFSTCDNQKSCMSNCSITSICEKPQEVCVAVWRKNDENITLETVC
HDPKLPYHDFILEDAASPKCIMKEKKKPGETFFMCSCSSDECNDNIIFS
EEYNTSNPD);

(b) an amino acid sequence at least 85%, 86%, 87%, 88%, 89%, 90%, 91%, 92%, 93%, 94%, 95%, 96%, 97%, 98%, or 99% identical to SEQ ID NO: 7; or

(c) an amino acid sequence with addition, deletion and/or substitution of one or more amino acids compared with SEQ ID NO: 7.

Method of Preparation

The present disclosure provides isolated nucleic acids or polynucleotides that encode the polypeptide complex, and the bispecific polypeptide complex provided herein.

The term “nucleic acid” or “polynucleotide” as used herein refers to deoxyribonucleic acids (DNA) or ribonucleic acids (RNA) and polymers thereof in either single- or double-stranded form. Unless specifically limited, the term encompasses polynucleotides containing known analogues of natural nucleotides that have similar binding properties as the reference nucleic acid and are metabolized in a manner similar to naturally occurring nucleotides. Unless otherwise indicated, a particular polynucleotide sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19: 5081 (1991); Ohtsuka et al., J. Biol. Chem. 260: 2605-2608 (1985); and Rossolini et al., Mol. Cell. Probes 8: 91-98 (1994)).

The nucleic acids or polynucleotides encoding the polypeptide complex and the bispecific polypeptide complex provided herein can be constructed using recombinant techniques. To this end, DNA encoding an antigen-binding moiety of a parent antibody (such as CDR or variable region) can be isolated and sequenced using conventional procedures (e.g., by using oligonucleotide probes that are capable of binding specifically to genes encoding the heavy and light chains of the antibody). Likewise, DNA encoding a TCR constant region can also be obtained. As an example, the polynucleotide sequence encoding the variable domain (VH) and the polynucleotide sequence encoding the first TCR constant region are obtained and operably linked to allow transcription and expression in a host cell to produce the first polypeptide. Similarly, polynucleotide sequence encoding VL are operably linked to polynucleotide sequence encoding second TCR constant region, so as to allow expression of the second polypeptide in the host cell. If needed, encoding polynucleotide sequences for one or more spacers are also operably linked to the other encoding sequences to allow expression of the desired product.

The encoding polynucleotide sequences can be further operably linked to one or more regulatory sequences, optionally in an expression vector, such that the expression or production of the first and the second polypeptides is feasible and under proper control.

The encoding polynucleotide sequence (s) can be inserted into a vector for further cloning (amplification of the DNA) or for expression, using recombinant techniques known in the art. In another embodiment, the polypeptide complex and the bispecific polypeptide complex provided herein may be produced by homologous recombination known in the art. Many vectors are available. The vector components generally include, but are not limited to, one or more of the following: a signal sequence, an origin of replication, one or more marker genes, an enhancer element, a promoter (e.g. SV40, CMV, EF-1α), and a transcription termination sequence.

The term “vector” as used herein refers to a vehicle into which a polynucleotide encoding a protein may be operably inserted so as to bring about the expression of that protein. Typically, the construct also includes appropriate regulatory sequences. For example, the polynucleotide molecule can include regulatory sequences located in the 5′-flanking region of the nucleotide sequence encoding the guide RNA and/or the nucleotide sequence encoding a site-directed modifying polypeptide, operably linked to the coding sequences in a manner capable of expressing the desired transcript/gene in a host cell. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating.

In some embodiments, the vector system includes mammalian, bacterial, yeast systems, etc., and comprises plasmids such as, but not limited to, pALTER, pBAD, pcDNA, pCal, pL, pET, pGEMEX, pGEX, pCI, pCMV, pEGFP, pEGFT, pSV2, pFUSE, pVITRO, pVIVO, pMAL, pMONO, pSELECT, pUNO, pDUO, Psg5L, pBABE, pWPXL, pBI, p15TV-L, pProl8, pTD, pRS420, pLexA, pACT2.2 etc., and other laboratorial and commercially available vectors. Suitable vectors may include, plasmid, or viral vectors (e.g., replication defective retroviruses, adenoviruses and adeno-associated viruses).

Vectors comprising the polynucleotide sequence (s) provided herein can be introduced to a host cell for cloning or gene expression. The phrase “host cell” as used herein refers to a cell into which an exogenous polynucleotide and/or a vector has been introduced.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for the vectors encoding the polypeptide complex and the bispecific polypeptide complex. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12, 424), K. bulgaricus (ATCC 16, 045), K. wickeramii (ATCC 24, 178), K. waltii (ATCC 56, 500), K. drosophilarum (ATCC 36, 906), K. thermotolerans, and K. marxianus; yarrowia (EP 402, 226); Pichia pastoris (EP 183, 070); Candida; Trichoderma reesia (EP 244, 234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated polypeptide complex, the bispecific polypeptide complex provided herein are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36: 59 (1977)), such as Expi293; baby hamster kidney cells (BHK, ATCC CCL 10); Chinese hamster ovary cells/-DHFR (CHO, Urlaub et al., Proc. Natl. Acad. Sci. USA 77: 4216 (1980)); mouse sertoli cells (TM4, Mather, Biol. Reprod. 23: 243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N. Y Acad. Sci. 383: 44-68 (1982)); MRC 5 cells; FS4 cells; and a human hepatoma line (Hep G2).

Host cells are transformed with the above-described expression or cloning vectors can be cultured in conventional nutrient media modified as appropriate for inducing promoters, selecting transformants, or amplifying the cloning vectors.

For production of the polypeptide complex and the bispecific polypeptide complex provided herein, the host cells transformed with the expression vector may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58: 44 (1979), Barnes et al., Anal. Biochem. 102: 255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

In certain embodiments, the polypeptide complex or the bispecific polypeptide complex may be linked to a conjugate indirectly, or indirectly for example through another conjugate or through a linker. For example, the polypeptide complex or the bispecific polypeptide complex having a reactive residue such as cysteine may be linked to a thiol-reactive agent in which the reactive group is, for example, a maleimide, an iodoacetamide, a pyridyl disulphide, or other thiol-reactive conjugation partner (Haugland, 2003, Molecular Probes Handbook of Fluorescent Probes and Research Chemicals, Molecular Probes, Inc.; Brinkley, 1992, Bioconjugate Chem. 3: 2; Garman, 1997, Non-Radioactive Labelling: A Practical Approach, Academic Press, London; Mean s (1990) Bioconjugate Chem. 1: 2; Hermanson, G in Bioconjugate Technique s (1996) Academic Press, San Diego, pp. 40-55, 643-671).

For another example, the polypeptide complex or the bispecific polypeptide complex may be conjugated to biotin, then indirectly conjugated to a seco nd conjugate that is conjugated to avidin. For still another example, the polypeptide complex or the bispecific polypeptide complex may be linked to a linker which further links to the conjugate. Examples of linkers include bifunctional coupling agents such as N-succinimidyl-3-(2-pyridyldithio) propionate (SPDP), succinimidyl-4- (N-maleimidomethyl) cyclohexane-1-carboxylate (SMCC) iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2, 6-diisocyanate), and his-active fluorine compounds (such as 1, 5-difluoro-2, 4-dinitrobenzene). Particularly preferred coupling agents include N-succinimidyl-3- (2-pyridyldithio) propionate (SPDP) (Carlsson et al., Biochem. J. 173: 723-737 (1978)) and N-succinimidyl-4- (2-pyridylthio) pentanoate (SPP) to provide for a disulphide linkage.

The conjugate can be a detectable label, a pharmacokinetic modifying moiety, a purification moiety, or a cytotoxic moiety. Examples of detectable lab el may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or β-D-galactosidase), radioisotopes (e.g. 1231, 124I, 125I, 131I, 35S, 3H, 111In, 112In, 14C, 64Cu, 67Cu, 86Y, 88Y, 90Y, 177Lu, 211At, 18 6Re, 188Re, 153Sm, 212Bi, and 32P, other lanthanides, luminescent labels), chromophoric moiety, digoxigenin, biotin/avidin, a DNA molecule or gold for detection. In certain embodiments, the conjugate can be a pharmacokinetic modifying moiety such as PEG which helps increase half-life of the antibody. Other suitable polymers include, such as, carboxymethylcellulose, dextran, polyvinyl alcohol, polyvinyl pyrrolidone, copolymers of eth ylene glycol/propylene glycol, and the like. In certain embodiments, the conjugate can be a purification moiety such as a magnetic bead. A “cytotoxic moiety” can be any agent that is detrimental to cells or that can damage or kill cells. Examples of cytotoxic moiety include, without limitation, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine) alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Methods for the conjugation of conjugates to proteins such as antibodies immunoglobulins or fragments thereof are found, for example, in U.S. Pat. Nos. 5,208,020; 6,441,163; WO2005037992; WO2005081711; and WO2006/034488, which are incorporated herein by reference to the entirety.

Pharmaceutical Composition

The present disclosure also provides a pharmaceutical composition comprising the polypeptide complex or the bispecific polypeptide complex provided herein and a pharmaceutically acceptable carrier.

The term “pharmaceutically acceptable” indicates that the designated carrier, vehicle, diluent, excipient (s), and/or salt is generally chemically and/or physically compatible with the other ingredients comprising the formulation, and physiologically compatible with the recipient thereof.

A “pharmaceutically acceptable carrier” refers to an ingredient in a pharmaceutical formulation, other than an active ingredient, which is bioactivity acceptable and nontoxic to a subject. Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Method of Treatment

Therapeutic methods are also provided, comprising: administering a therapeutically effective amount of the polypeptide complex or the bispecific polypeptide complex provided herein to a subject in need thereof, thereby treating or preventing a condition or a disorder. In certain embodiments, the subject has been identified as having a disorder or condition likely to respond to the polypeptide complex or the bispecific polypeptide complex provided herein.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc. Except when noted, the terms “patient” or “subject” are used interchangeably.

The terms “treatment” and “therapeutic method” refer to both therapeutic treatment and prophylactic/preventative measures. Those in need of treatment may include individuals already having a particular medical disorder as well as those who may ultimately acquire the disorder.

In certain embodiments, the conditions and disorders include tumors and cancers, for example, non-small cell lung cancer, small cell lung cancer, renal cell cancer, colorectal cancer, ovarian cancer, breast cancer, pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphomas, myelomas, mycoses fungoids, merkel cell cancer, and other hematologic malignancies, such as classical Hodgkin lymphoma (CHL), primary mediastinal large B-cell lymphoma, T-cell/histiocyte-rich B-cell lymphoma, EBV-positive and -negative PTLD, and EBV-associated diffuse large B-cell lymphoma (DLBCL), plasmablastic lymphoma, extranodal NK/T-cell lymphoma, nasopharyngeal carcinoma, and HHV8-associated primary effusion lymphoma, Hodgkin's lymphoma, neoplasm of the central nervous system (CNS), such as primary CNS lymphoma, spinal axis tumor, brain stem glioma.

### EXAMPLES

### Example 1: Research Materials Preparation

1. PD-L1 Antigen and TGFβ Antigen Preparation

Human PD-L1 extracellular domain (ECD) antigen His tagged was purchased from Sino Biological (Cat#10084-H08H). Mouse PD-L1 ECD antigen His tagged was purchased from Sino Biological (Cat#50010-M08H). Cynomolgus monkey (cyno) PD-L1 ECD antigen His tagged was purchased from Sino Biological (Cat#90251-C08H). Human TGFβ1, TGFβ2 and TGFβ3 antigens were purchased from R&D Systems (Cat#7754-BH, 7754-BH/CF; Cat#302-B2, 302-B2/CF; Cat#8420-B3, 8420-B3/CF).

2. Establishment of PD-L1 Expressing Cell Lines

Human PD-L1 expressing cell line (W315-CHO-K1. hPro1. C11), mouse PD-L1-expressing cell line (W315-293F. mPro1. C1) and cynomolgus monkey PD-L1-expressing cell line (W315-293F. cynoPro1.2A2) were generated in house. Using Lipofectamine 2000 (ThermoFisher-11668027), CHO-K1 or 293F cells were transfected with the expression vector containing gene encoding full length human PD-L1 or cynoPD-L1 or mouse PD-L1. Cells were cultured in medium containing proper selection pressure. The stable cell lines were obtained by limiting dilution.

3. Production of Benchmark Antibody (BMK) and Control Antibody (cAb)

TGFβRII ECD fusion anti-PD-L1 BMK antibody named as WT112-BMK2-IgG1 was constructed based on the sequence of M7824 in U.S. Pat. No. 9,676,863B2 from Merck Patent GmbH. The plasmid containing heavy chain gene and plasmid containing light chain gene were co-transfected to Expi293 cells with Expi293 expression kit (ThermoFisher-A14524).

The TGFβRII ECD fusion control antibody WT112-cAb2 was constructed in the same way as WT112-BMK2-IgG1. The antibody part of WT112-cAb2 was human IgG1 isotype control antibody; the linker between the Fc and TGFβRII ECD was (G4S) 4; the TGFβRII ECD sequence was the same as that in WT112-BMK2-IgG1.

4. Production of WT1126-U15T1. G1-1. uIgG1

WT1126-U15T1. G1-1. uIgG1 is an anti-PD-L1 VHH Fc fusion antibody fused with TGFβRII ECD. The DNA encoding anti-PD-L1 VHH variable region was insert into modified pcDNA3.3 expression vectors containing Fc of human IgG1. The C-terminus of Fc was the sequence encoding TGFβRII ECD with a (G4S) 4 linker in between, which was as the same as that in WT112-BMK2-IgG1. The schematic diagram of WT1126-U15T1. G1-1. uIgG1 is shown in FIG. 1.

### Example 2: In Vitro Characterization

1. Human TGF-β Binding ELISA

The binding of antibodies to human TGF-β1, TGF-β2 and TGF-β3 was determined by ELISA. Plates were coated with human TGF-β1, TGF-β2 or TGF-β3 overnight at 4° C., respectively. After blocking and washing, various concentrations of lead antibodies were added to the plates and incubated at room temperature for 1 hour. The plates were then washed and incubated with HRP-labeled goat anti-human IgG antibody (Bethyl) for 1 hour. After washing, TMB substrate was added and the color reaction was stopped by 2M HCl. The absorbance at 450 nm and 540 nm was read using a microplate reader (SpectraMax M5e).

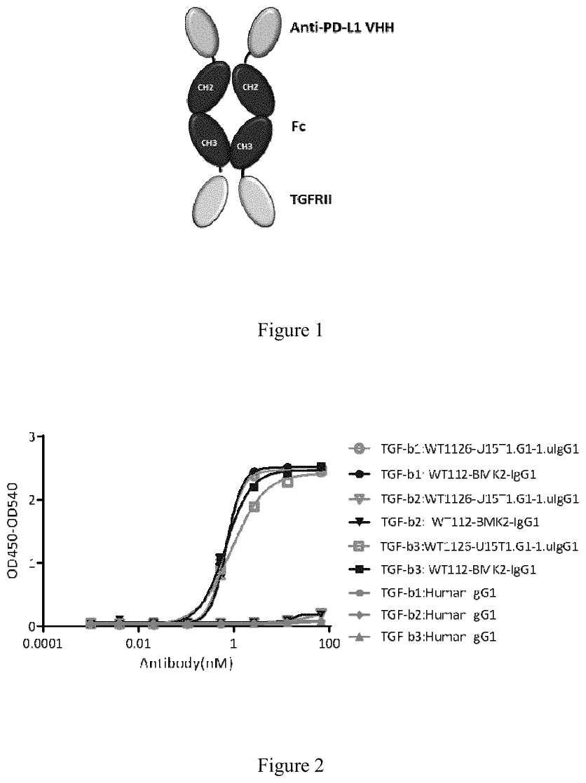

The binding curves of antibodies to plate coated human TGF-β1, TGF-β2 and TGF-β3 were shown in FIG. 2. WT1126-U15T1. G1-1. uIgG1 shows similar affinity as WT112-BMK2-IgG1. They strongly bind to immobilized TGF-β1 (EC50=0.67 nM) and TGF-β3 (EC50=0.89 nM) but not to immobilized TGF-β2.

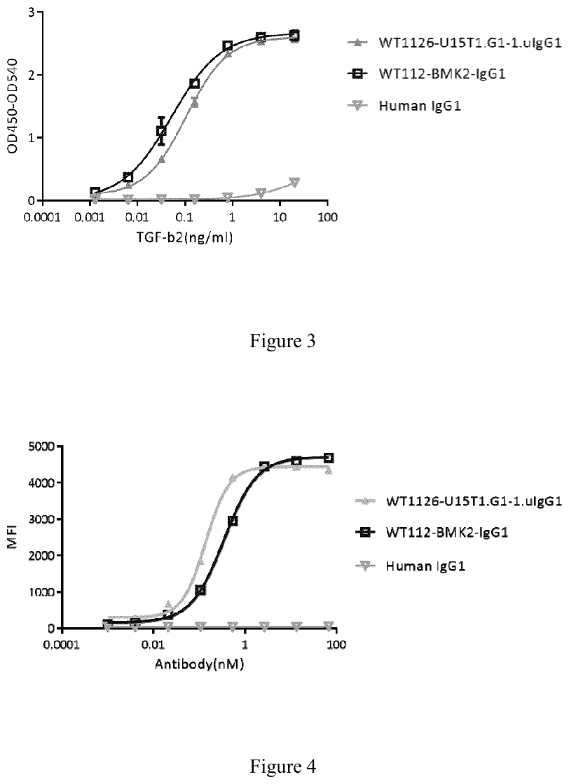

The binding of antibody to human TGF-β2 was also determined by ELISA immobilized with testing antibody. After blocking and washing, various concentrations of TGF-β2 were added to the plates and incubated at room temperature for 1 hour. The plates were then washed and incubated with biotinylated TGF-β2 detection antibody (R&D, DY240) for 1 hour, followed by streptavidin-HRP for 1 hour. After washing, TMB substrate was added and the color reaction was stopped by 2M HCl. The absorbance at 450 nm and 540 nm was read using a microplate reader (SpectraMax M5e). The binding curves of antibodies to soluble human TGF-β2 were shown in FIG. 3. Immobilized WT1126-U15T1. G1-1. uIgG1 and WT112-BMK2-IgG1 can bind to soluble TGF-β2 with comparable EC 50 of 0.11 nM and 0.05 nM, respectively.

2. Human PD-L1 Binding FACS

Various concentrations of testing antibodies were incubated with hPD-L1 expressing W315-CHO-K1. hPro1. C11 cells at 4° C. for 1 hour. After washing, the cells were incubated with PE-labeled goat anti-human IgG-Fc antibody (Jackson Immuno Research). Finally, the MFI of the cells was measured by a flow cytometer and analyzed by FlowJo. The binding curves to human PD-L1 transfected cells are shown in FIG. 4. WT1126-U15T1. G1-1. uIgG1 and WT112-BMK2-IgG1 strongly bind to cell surface human PD-L1 with EC 50 of 0.14 nM and 0.35 nM, respectively.

3. Cross Species Binding FACS

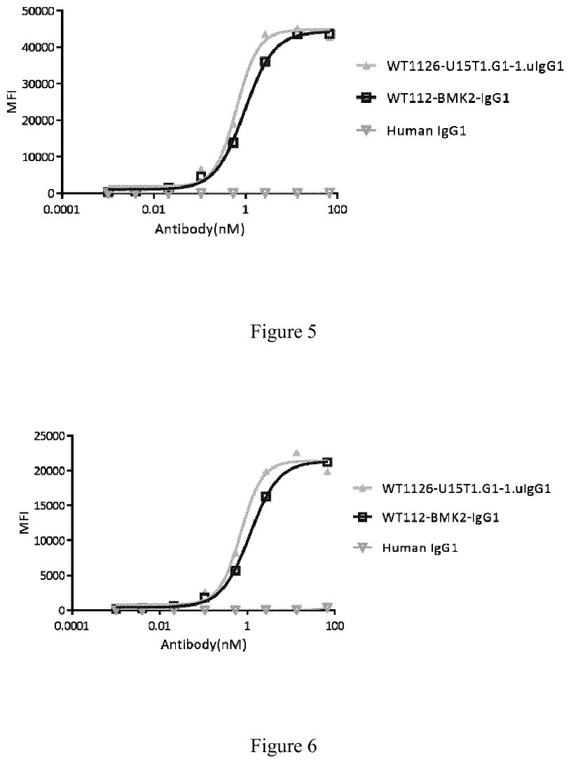

The binding of testing antibody to cynomolgus monkey or mouse PD-L1 was determined by FACS. Various concentrations of testing antibody were incubated with cynomolgus PD-L1-expressing W315-293F. cynoPro1.2A2 cells or mouse PD-L1 expressing W315-293F. mPro1. C1 cells at 4° C. for 1 hour, and then the binding of antibodies to the surface of the cells was detected by PE-labeled goat anti-human IgG-Fc antibody (Jackson Immuno Research). MFI of the cells was measured by a flow cytometer and analyzed by FlowJo. The binding to cyno PD-L1 transfected cells was shown in FIG. 5 and to mouse PD-L1 transfected cells in FIG. 6. WT1126-U15T1. G1-1. uIgG1 and WT112-BMK2-IgG1 can strongly bind to cell surface cyno and mouse PD-L1. The cyno PD-L1 binding EC 50 of WT1126-U15T1. G1-1. uIgG1 and WT112-BMK2-IgG1 are 0.63 nM and 0.96 nM, respectively. The mouse PD-L1 binding EC 50 are 0.71 nM and 1.3 nM, respectively.

4. Simultaneous Binding with Human PD-L1 and Human TGF-β1

The simultaneous binding of testing antibody to human TGF-β1 and human PD-L1 was determined by ELISA. Plates were coated with human TGF-β1 overnight at 4° C. After blocking and washing, various concentrations of testing antibodies were added to the plates and incubated at room temperature for 1 hour. The plates were then washed and incubated with biotinylated human PD-L1 ECD protein followed by streptavidin-HRP (Invitrogen) for 1 hour. After washing, TMB substrate was added and the color reaction was stopped by 2M HCl. The absorbance at 450 nm and 540 nm was read using a microplate reader (SpectraMax M5e). Similarly, the simultaneous dual target binding was also tested by coating the plate with human PD-L1. After incubation with various concentrations of testing antibodies and then TGF-β1 antigen, the biotinylated human TGF-β1 detecting antibody (R&D, Cat 840117) and followed by streptavidin-HRP (Invitrogen) was added in the plate. Finally, TMB substrate was added and the color reaction was stopped by 2M HCl. The absorbance at 450 nm and 540 nm was read using a microplate reader (SpectraMax M5e).

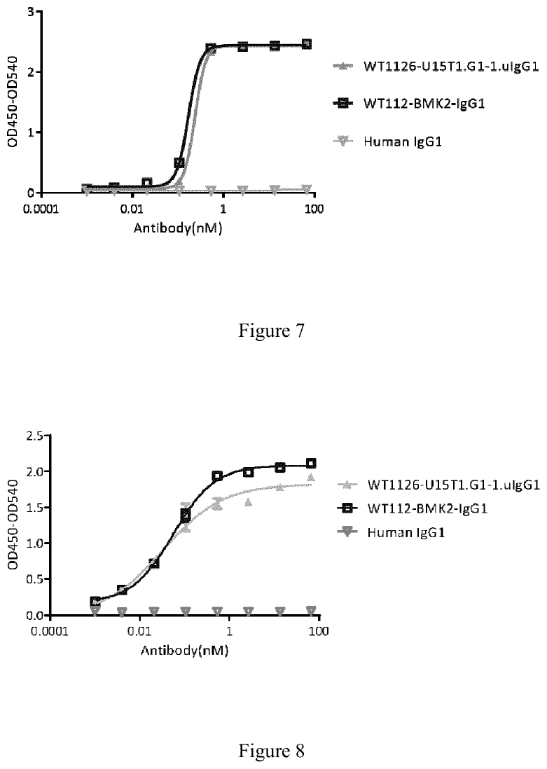

The result shown in FIG. 7 and FIG. 8 indicated that WT1126-U15T1. G1-1. uIgG1 and WT112-BMK2-IgG1 simultaneously bind to PD-L1 and TGF-β1 with EC 50 of 0.24 and 0.17 nM respectively when TGF-β1 was immobilized; EC 50 of 0.03 nM and 0.06 nM respectively when the antibodies were immobilized.

5. PD-1/PD-L1 Blockade by Competition FACS

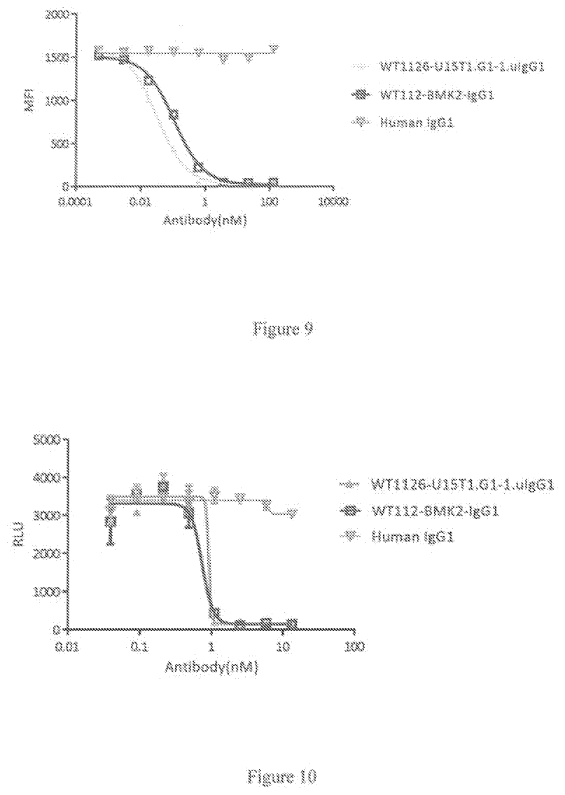

Various concentrations of lead antibody, positive and negative control antibodies were mixed with mFc tagged human PD-1, and then incubated with human PD-L1 expressing transfected cells at 4° C. for 1 hour. The binding of human PD-1 to human PD-L1 expressing cells was detected by PE-labeled anti-mouse IgG Fc antibody (Abcam). MFI of the cells was measured by a flow cytometer and analyzed by FlowJo. As shown in FIG. 9, WT1126-U15T1. G1-1. uIgG1 and WT112-BMK2-IgG1 block the binding of PD-1 to cell surface PD-L1 with IC 50 of 0.03 nM and 0.11 nM, respectively.

6. Reporter Gene Assay (RGA)

The blockade of TGF-β1 signaling was tested by a RGA assay. The RGA cell line was made by stably expressing full length of human Activin Receptor II B along with stably integrated SBE luciferase reporter gene. To test TGF-β1 signaling blockade activity of testing antibody, human TGF-β1 and various concentrations of antibodies were pre-mixed and added into the RGA cells and incubated overnight at 37° C., 5% CO 2. After incubation, reconstituted luciferase substrate (Promega, Cat E6130) was added and the luciferase intensity was measured by a microplate spectrophotometer. WT1126-U15T1. G1-1. uIgG1 displays comparable TGF-β1 blockade IC 50 of 0.7 nM to WT112-BMK2 (IC 50=0.7 nM) as shown in FIG. 10.

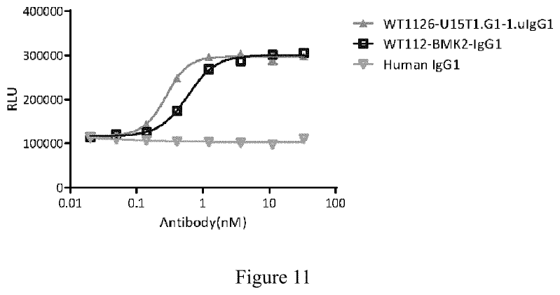

The blockade of PD-1/PD-L1 signaling was test by a RGA assay. PD-1 RGA cell line was made by stably expressing full length of PD-1 along with NFAT luciferase reporter gene in Jurkat E6-1 cells. The PD-1 RGA cells were incubated with human PD-L1 expressing artificial APC (ahuman PD-L1 and OKT3 sc-Fv expressing CHO-Ki cells) in the presence of various concentrations of testing antibodies for 4-6 hours at 37° C., 5% CO 2. After incubation, reconstituted luciferase substrate was added and the luciferase intensity was measured by a microplate spectrophotometer. As show in FIG. 11, WT1126-U15T1. G1-1. uIgG1 and WT112-BMK2-IgG1 show strong hPD-1/PD-L1 signaling blockade activity in the RGA assay. The IC50 of WT1126-U15T1. G1-1. uIgG1 and WT112-BMK2-IgG1 are 0.28 and 0.59 nM, respectively.

7. Allogeneic Mixed Lymphocyte Reaction (Allo-MLR)

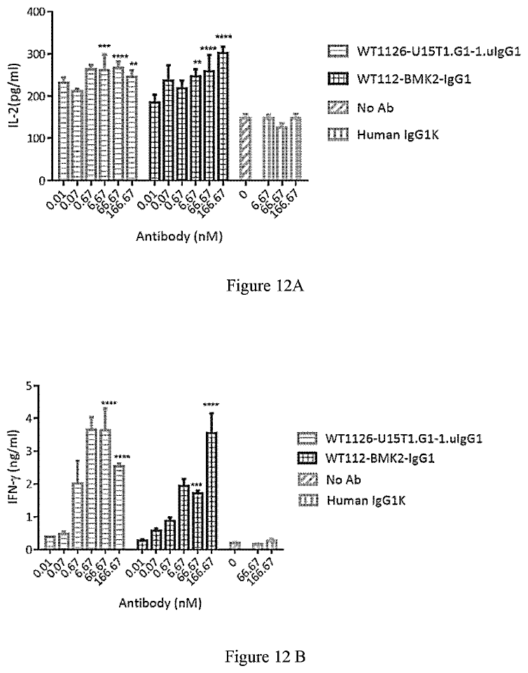

Human peripheral blood mononuclear cells (PBMCs) were freshly isolated from healthy donors using Ficoll-Paque PLUS (Stem Cell) gradient centrifugation. Monocytes were isolated using CD14 MicroBeads (Miltenyi Biotec) according to the manufacturer's instructions. Cells were cultured in medium containing GM-CSF (Amoytop Biotech) and IL-4 (R&D) for 5 to 7 days to generate dendritic cells (DC). Human CD4+ T cells were isolated using human CD4+ T cell enrichment kit (Stem Cell) according to the manufacturer's protocol. Purified CD4+ T cells were co-cultured with allogeneic immature DCs (iDCs) in the presence of various lead antibody, positive and negative control antibodies in 96-well plates. The plates were incubated at 37° C., 5% CO 2. Supernatants were harvested for IL-2 and IFN-γ test at day 3 and day 5, respectively. Human IL-2 and IFN-γ release were measured by ELISA using matched antibody pairs. Recombinant human IL-2 (R&D) and IFN-γ(PeproTech) were used as standards, respectively. The plates were pre-coated with capture antibody specific for human IL-2 (R&D) or IFN-γ (Pierce), respectively. After blocking, 50 μL of standards or samples were pipetted into each well and incubated for 2 hours at ambient temperature. Following removal of the unbound substances, the biotin-conjugated detecting antibody specific for corresponding cytokine was added to the wells and incubated for one hour. HRP-labeled streptavidin was then added to the wells for 30 minutes incubation at ambient temperature. The color was developed by dispensing 50 μL of TMB substrate, and then stopped by 50 μL of 2N HCl. The absorbance was read at 450 nm and 540 nm using a microplate spectrophotometer. The results shown in FIG. 12 demonstrate that WT1126-U15T1. G1-1. uIgG1 and WT112-BMK2-IgG1 can enhance the IL-2 production (FIG. 12A) and IFNγproduction (FIG. 12B) in human CD4+ T cells allo-MLR assay in a dose dependent manner. (Analyzed by two-way ANOVA vs isotype control. *p<0.05; **p<0.01; ***p<0.001, ****p<0.0001)

8. Serum Stability

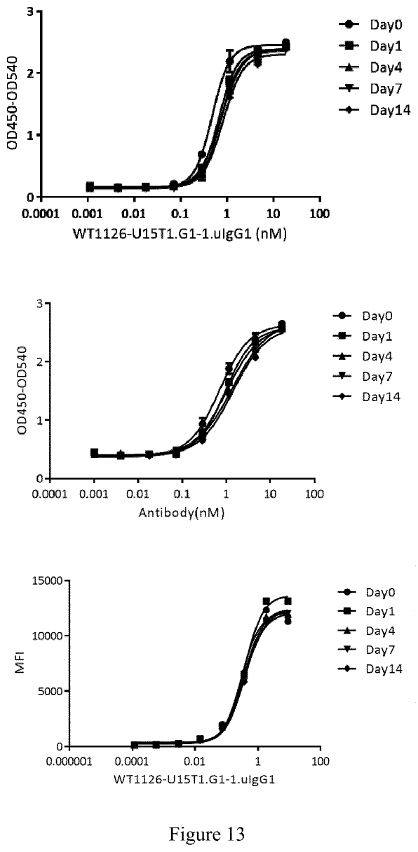

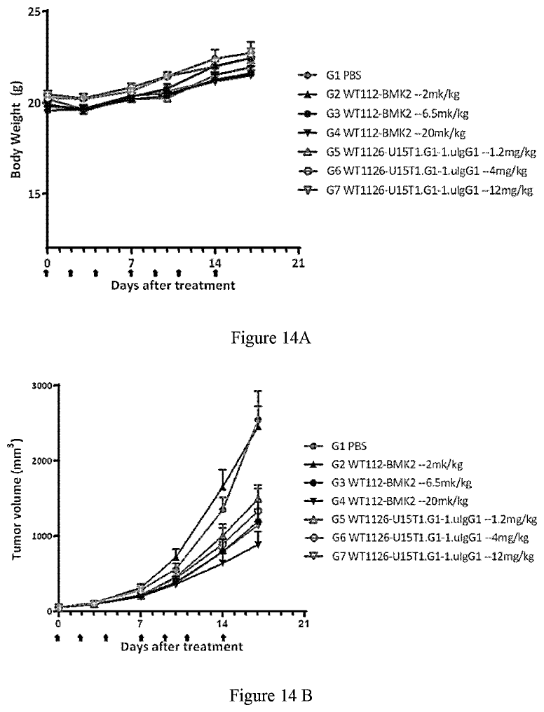

WT1126-U15T1. G1-1. uIgG1 was incubated in freshly isolated human serum (serum content>95%) at 37° C. in a 5% CO 2 incubator. At indicated time point, aliquot of serum treated samples were removed from the incubator and snap frozen in liquid N 2, and then stored at 20° C. until ready for test. The samples were quickly thawed immediately prior to the stability test. The procedure of simultaneous binding ELISA, human TGF-β1 binding ELISA and human PD-L1 binding FACS were described above. As shown in FIG. 13, these WT1126-U15T1. G1-1. uIgG1 samples show normal binding to targets, suggesting that the antibody is stable in human serum for at least 14 days.

9. Antibody Protein Accelerated Stability Study

9.1 Sample Treatment and Accelerated Stability Study

WT1126-U15T1. G1-1. uIgG1 was dialyzed via dialysis bag (Spectrum-888-10987, MWCO 3.5 kDa) into PBS buffer and then diluted to 2 ug/ml. An accelerated stability study was conducted by incubation of testing antibody at 4° C., 25° C. and 40° C. respectively for 1 day, 4 days and 7 days, as well as freeze-thawed for 3 cycles at −80° C. (Table 2). After incubation at each testing condition, the sample visual inspection was performed immediately to carefully detect the presence of any particulates. All the samples appeared as clear solution free of particulates. The antibody stability of each treated sample was analyzed by SDS-PAGE, analytic SEC-HPLC, DSF and DLS assays. The samples after different treatments are stable at DSF and DLS analyses compared with the sample freshly thawed from −80° C. (TO in table 2). SEC-HPLC analysis indicated that low molecule weight percentage (LMW %) was increased after incubation at 40° C. for 4 days and 7 days in PBS buffer (Table 2). After formulation buffer optimization, the antibody appear stable at 40° C. in sodium acetate buffer (Table 3).

9.2 Thermo Stability by DSF

A DSF assay was performed using Real-Time Fluorescent Quantitative PCR (QuantStudio 7 Flex, Thermo Fisher Scientific). Briefly, 19 μL of antibody solution was mixed with 1 μL of 62.5×SYPRO Orange solution (Invitrogen) and added to a 96 well plate (Biosystems). The plate was heated from 26° C. to 95° C. at a rate of 0.9° C./min, and the resulting fluorescence data were collected. The negative derivatives of the fluorescence changes with respect to different temperatures were calculated, and the maximal value was defined as melting temperature. Data collection and Tm calculation was conducted automatically by operation software (QuantStudio™ Real-Time PCR Software v1.3). The Tm of WT1126-U15T1. G1-1. uIgG1 in PBS buffer is about 65° C. (Table 2).

9.3 Molecule Radius Measurement by DLS

Molecule radius measurement was investigated using DynaPro Plate Reader III dynamic light scattering (DLS) instrument (Wyatt Dynapro™). Five acquisitions were collected for each protein sample and each acquisition time was 5 s. Each well contained 7.5 μL of solution in 1536 plate (Aurora microplate). For each measurement, the diffusion coefficient was determined. Radius were calculated automatically by the operation software (DYNAMICS 7.8.1.3). The results shown in table 2 suggested that the radius ranges of samples after different treatments are from 25.9 nM to 33.5 nM, which are comparable to the sample freshly thawed from −80° C. (Radius of TO is 28 nM).

TABLE 2
Accelerated stability result in PBS.

| | | | DLS | SEC (HMW/ | |
|---|---|---|---|---|---|
| | | Conc. | Radius | Mono/LMW | DSF |
| Treatment | Appearance | (mg/mL) | (nm) | %) | Tm ° C. |
| T0 | particle-free | 2.04 | 28.0 | 5.01/94.98% | 64.6 |
| 3X | particle-free | 1.85 | 30.2 | 5.23/93.29/ | 65.1 |
| | | | | 1.49% | |
| 4° C.-1 | particle-free | 1.90 | 33.5 | 4.87/93.62/ | 64.8 |
| Day | | | | 1.51% | |
| 4° C.-4 | particle-free | 1.86 | 29.0 | 4.75/93.93/ | 64.9 |
| Days | | | | 1.33% | |
| 4° C.-7 | particle-free | 1.85 | 27.8 | 4.52/95.17/ | 65.1 |
| Days | | | | 0.31% | |
| 25° C.-1 | particle-free | 1.86 | 27.9 | 4.73/93.85/ | 65.1 |
| Day | | | | 1.25% | |
| 25° C.-4 | particlc-frcc | 1.81 | 29.2 | 4.63/94.06/ | 64.9 |
| Days | | | | 1.32% | |
| 25° C.-7 | particle-free | 1.85 | 28.3 | 4.38/95.29/ | 64.9 |
| Days | | | | 0.32% | |
| 40° C.-1 | particlc-frcc | 1.85 | 28.7 | 4.42/95.58% | 64.9 |
| Day | | | | | |
| 40° C.-4 | particle-free | 1.84 | 25.9 | 4.64/86.85/ | 64.8 |
| Days | | | | 8.51% | |
| 40° C.-7 | particle-free | 1.82 | 32.5 | 4.92/81.47/ | 64.6 |
| Days | | | | 13.16% | |

T0: It has been frozen and thawed once (from −80° C.).
3X: The sample was frozen and thawed for 3 more times than T0.

TABLE 3
Accelerated stability test at 40° C. in sodium acetate buffer

| | | Conc. | SEC (HMW/Mono/ |
|---|---|---|---|
| Treatment | Formulation | mg/mL | LMW %) |
| T0 | 20 mM Sodium acetate, | 2.07 | 1.34/98.67% |
| 40° C.-l Day | 7% Sucrose, | 2.04 | 1.10/98.89% |
| 40° C.-4 Days | 0.02% PS80 pH 5.0 | 2.02 | 1.00/98.87/0.13% |
| 40° C.-7 Days | | 2.03 | 1.05/98.65/0.29% |
| T0 | 20 mM Sodium acetate, | 1.97 | 1.47/98.53% |
| 40° C.-1 Day | 7% Sucrose, | 1.96 | 1.21/98.79% |
| 40° C.-4 Days | 135 mM Arginine | 1.96 | 1.03/98.97% |
| 40° C.-7 Days | 0.02% PS80, pH 5.0 | 1.96 | 1.05/98.65/0.29% |

10. Full Kinetic Binding Affinity to PD-L1 by SPR

WT1126-U15T1. G1-1. uIgG1 binding affinity to human, mouse and cyno PD-L1 was detected by SPR assay using Biacore 8K. Antibody was captured on an anti-human IgG Fc antibody immobilized CM5 sensor chip (GE). Human or cyno PD-L1 at different concentrations were injected over the sensor chip at a flow rate of 30 μL/min for an association phase of 180 s, followed by 3600 s dissociation. Mouse PD-L1 at different concentrations were injected over the sensor chip at a flow rate of 30 t/min for an association phase of 120 s, followed by 1200 s dissociation. The chip was regenerated by 10 mM glycine (pH 1.5) after each binding cycle.

The sensorgrams of blank surface and buffer channel were subtracted from the test sensorgrams. For WT1126-U15T1. G1-1. uIgG1 binding to mouse PD-L1, 0-300 s curves were used in the fitting process. The experimental data was fitted by 1:1 model using Langmiur analysis. Molecular weight of 40 kDa was used to calculate the molar concentration of human, mouse and cyno PD-L1. As shown in Table 4, WT1126-U15T1. G1-1. uIgG1 has similar affinity to human and cyno PD-L1.

TABLE 4
Binding affinity of WT1126-U15T1.G1-1.uIgG1 to human, cyno and
mouse PD-L1

| Analyte | Ligand | ka (1/Ms) | kd (1/s) | KD (M) |
|---|---|---|---|---|
| Human | WT1126-U15T1.1.uIgG1 | 1.15E+06 | 3.07E−04 | 2.67E−10 |
| PD-L1 | | | | |
| Cyno | WT1126-U15T1.1.uIgG1 | 1.01E+06 | 4.65E−04 | 4.61E−10 |
| PD-L1 | | | | |
| Mouse | WT1126-U15T1.1.uIgG1 | 2.58E+05 | 1.08E−02 | 4.19E−08 |
| PD-L1 | | | | |

11. Full Kinetic Binding Affinity to FcRn by SPR

Antibody binding affinity to human FcRn (ARCO, FCM-H5286) was detected using Biacore 8K. Each antibody was immobilized on CM5 sensor chip (GE). Human FcRn at different concentrations were injected over the sensor chip at a flow rate of 30 uL/min for an association phase of 60 s, followed by 90 s dissociation. The chip was then regenerated by 10 mM glycine (pH 1.5) after each binding cycle. The sensorgrams of blank surface and buffer channel were subtracted from the test sensorgrams. The experimental data was fitted by steady-state affinity model. Molecular weight of 45 KDa was used to calculate the molar concentration of analyte FcRn. The running buffer was PBST, pH 6.0.

The affinities of WT1126-U15T1. G1-1. uIgG1 and WT112-BMK2-IgG1 to FcRn are similar (Table 5).

TABLE 5
Affinity to FcRn by SPR

| Analyte | Ligand | K_{D} (M) |
|---|---|---|
| Human FcRn | WT1126-U15T1.G1.uIgG1 | 1.47E−06 |
| | WT112-BMK2-IgG1 | 1.89E−06 |

### Example 3. In Vivo Antitumor Efficacy Study

WT1126-U15T1. G1-1. uIgG1 antitumor efficacy study was tested in CT26 model in BALB/C female mice. Female BALB/C mice (Zhejiang Vital River Lab Animal Technology Co. Ltd) of 8 week-old were used in the study. BALB/C cells were maintained in vitro as a monolayer culture in RPMI 1640 medium supplemented with 10% fetal bovine serum, 100 U/mL penicillin and 100 μg/mL streptomycin at 37° C. in an atmosphere of 5% CO 2 in air. The tumor cells were routinely sub-cultured twice a week with 0.25% trypsin-EDTA treatment. The cells growing in an exponential growth phase were harvested and counted for tumor inoculation.

For the therapeutic model, each mouse was inoculated subcutaneously at the right rear flank with CT26 tumor cells (5.0×10 5 cells in 100 μL RPMI). When the average tumor volume reached approximately to 50 mm 3, animals were randomly grouped into 7 groups and each group contained 10 mice. The mice received injections intraperitoneally three times a week for total 7 injections. 6 groups (G2-G7) were injected with equal molar amounts of WT112-BMK2-IgG1 and WT1126-U15T1. G1-1. uIgG1 at low, mid and high dose levels, respectively. The control group (G1) received injections with vehicle-PBS. The dose levels for WT112-BMK2-IgG1 were 2 mg/kg, 6.5 mg/kg and 20 mg/kg; for WT1126-U15T1. G1-1. uIgG1 are 1.2 mg/kg, 4 mg/kg and 12 mg/kg, respectively. The day of the first injection was considered as day 0. For all tumor studies, mice were weighed and tumor growth was measured twice a week using calipers. All the procedures related to animal handling, care and the treatment in the study were performed according to the guidelines approved by the Institutional Animal Care and Use Committee (IACUC) of Shanghai Model Organisms Center, Inc., and following the guidance of the Association for Assessment and Accreditation of Laboratory Animal Care (AAALAC). Tumor volume was calculated with the formula (½ (length×width 2). The results were represented by mean and the standard error (Mean±SEM). Data were analyzed by Graphpad Prism 6.0 and p values analyzed by T test. p<0.05 was considered statistically significant.

As shown in FIG. 14A, all mice were normal during the experiment and slowly gained weight, suggesting that antibodies were not toxic. As shown in FIG. 14B, at the 17 days after the first dosing, the mean tumor volume of vehicle group was 2550 mm3, which indicated CT26 model was well established. Compared with vehicle group, WT1126-U15T1. G1-1. uIgG1 showed potent antitumor effect and significantly inhibited tumor growth. TGI (tumor growth inhalation) was calculated for each group using the formula: TGI (%)=[1−(Ti−T0)/(Vi−V0)]×100. Ti is the average tumor volume of a treatment group on a given day. TO is the average tumor volume of the treatment group on the first day of treatment. Vi is the average tumor volume of the vehicle control group on the same day with Ti and V0 is the average tumor volume of the vehicle group on the first day of treatment. The TGI at day 17 of each group was 3.67%, 54.26% and 66.46% for WT112-BMK2-IgG1 at dose level of 2 mg/kg, 6.5 mg/kg and 20 mg/kg, respectively. 42.21%, 48.69% and 56.05% for WT1126-U15T1. G1-1. uIgG1 at 1.2 mg/kg, 4 mg/kg and 12 mg/kg, respectively. WT1126-U15T1. G1-1. uIgG1 exhibited superior antitumor activity to BMK2 at low dose (p<0.01, by T-test of G2 vs G5) and comparable antitumor activity at mid. and high doses (p=no significance (ns) by T-test of G3 vs G6 and G4 vs G7).

1. A polypeptide, comprising, from N-terminus to C-terminus,
a) at least a variable region of a heavy chain of a heavy-chain antibody (VHH domain) that binds human, cynomolgus monkey, or mouse protein Programmed Death Ligand 1 (PD-L1), operably linked to IgG Fc binding domain; and
b) human TGFβRII, or a fragment thereof capable of binding TGFβ1.

2. The polypeptide of claim 1, wherein the VHH domain comprises one or more heavy chain CDRs (CDRHs) selected from at least one of the group consisting of:
(a) a CDRH1 with at least 70%, sequence identity to a CDRH1 as depicted in SEQ ID NO: 1;
(b) a CDRH2 with at least 70%, sequence identity to a CDRH2 as depicted in SEQ ID NO:2; and
(c) a CDRH3 with at least 70%, sequence identity to a CDRH3 as depicted in SEQ ID NO:3.

3. The polypeptide of claim 2, wherein the VHH domain comprises one or more heavy chain CDRs (CDRHs) selected from at least one of the group consisting of:
(a) a CDRH1 as depicted in SEQ ID NO:1 or a CDRH1 that differs in amino acid sequence from the CDRH1 by an amino acid addition, deletion or substitution of not more than 2 amino acids;
(b) a CDRH2 as depicted in SEQ ID NO:2 or a CDRH2 that differs in amino acid sequence from the CDRH2 by an amino acid addition, deletion or substitution of not more than 2 amino acids; and
(c) a CDRH3 as depicted in SEQ ID NO: 3 or a CDRH3 that differs in amino acid sequence from the CDRH3 by an amino acid addition, deletion or substitution of not more than 2 amino acids.

4. The polypeptide of claim 2, wherein the VHH domain comprises one or more heavy chain CDRs (CDRHs) selected from at least one of the group consisting of:
(a) a CDRH1 comprising or consisting of SEQ ID NO:1;
(b) a CDRH2 comprising or consisting of SEQ ID NO:2; and
(c) a CDRH3 comprising or consisting of SEQ ID NO:3.

5. The polypeptide of claim 2, wherein the VHH domain comprises:
(a) the amino acid sequence of SEQ ID NO:4;
(b) an amino acid sequence at least 85%, 90%, 95%, or 99% identical to SEQ ID NO:4; or
(c) an amino acid sequence with addition, deletion and/or substitution of one or more amino acids compared with SEQ ID NO:4.

6. The polypeptide of claim 1, further comprising an amino acid linker connecting the C-terminus of the VHH domain or IgG Fc binding domain to the N-terminus of the human TGFβRII or fragment thereof.

7. The polypeptide of claim 6, wherein the linker comprises:
(a) the amino acid sequence of SEQ ID NO:5;
(b) an amino acid sequence at least 85%, 90%, 95%, or 99% identical to SEQ ID NO:5; or
(c) an amino acid sequence with addition, deletion and/or substitution of one or more amino acids compared with SEQ ID NO:5.

8. The polypeptide of claim 1, wherein the human TGFβRII, or a fragment thereof comprises:
(a) the amino acid sequence of SEQ ID NO:6;
(b) an amino acid sequence at least 85%, 90%, 95%, or 99% identical to SEQ ID NO:6; or
(c) an amino acid sequence with addition, deletion and/or substitution of one or more amino acids compared with SEQ ID NO:6.

9. The polypeptide of claim 1, comprising:
(a) the amino acid sequence of SEQ ID NO:7;
(b) an amino acid sequence at least 85%, 90%, 95%, or 99% identical to SEQ ID NO:7; or
(c) an amino acid sequence with addition, deletion and/or substitution of one
or more amino acids compared with SEQ ID NO:7.

10. An antibody or antigen binding fragment thereof, wherein the antibody or antigen binding fragment thereof comprises two of said polypeptide of claim 1.

11. A nucleic acid molecule comprising a nucleotide sequence encoding a polypeptide according to claim 1.

12. A cloning or expression vector comprising the nucleic acid molecule of claim 11.

13. A cell comprising the nucleic acid of claim 11.

14. (canceled)

15. A pharmaceutical composition comprising the polypeptide of claim 1, and one or more of a pharmaceutically acceptable excipient, a diluent and a carrier.

16. (canceled)

17. A method of treating tumor or inhibiting growth of tumor cells in a subject, comprising administering to the subject a therapeutically effective amount of the polypeptide of claim 1.

18. (canceled)

19. The method of claim 17, wherein the tumor is selected from the group consisting of colorectal, breast, ovarian, pancreatic, gastric, prostate, renal, cervical, myeloma, lymphoma, leukemia, thyroid, endometrial, uterine, bladder, neuroendocrine, head and neck, liver, nasopharyngeal, testicular, small cell lung cancer, non-small cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodisplastic syndromes.
